(19) United States
(12) Patent Application Publication
(10) Pub. No.: US 2023/0211123 A1
(43) Pub. Date: Jul. 6, 2023

(54) MEASURING INJECTION CATHETER NEEDLE INSERTION DEPTH AND INJECTION EFFICACY

(71) Applicant: Chelak Medical Solution Inc., Houston, TX (US)

(72) Inventors: Jie CHENG, Bellaire, TX (US); Emerson PERIN, Houston, TX (US); Dhanunjaya LAKKIREDDY, Leawood, KS (US)

(21) Appl. No.: 18/090,329

(22) Filed: Dec. 28, 2022

Related U.S. Application Data
(63) Continuation of application No. PCT/US2021/039950, filed on Jun. 30, 2021.
(60) Provisional application No. 63/046,319, filed on Jun. 30, 2020.

(51) Int. Cl. A61M 25/00

(52) U.S. Cl. CPC A61M 25/0084; A61M 2025/0089; A61M 2205/3317; A61M 2205/3327; A61M 2210/125

(57) ABSTRACT

Certain aspects of the present disclosure provide methods and apparatus for measuring an injection catheter needle insertion depth and/or injection solution efficacy. An example injection catheter may include a catheter tube and a retractable, electrically conductive needle disposed in the catheter tube and configured to extend from the catheter tube. The injection catheter may also include one or more electrodes disposed at a distal portion of the catheter tube, an electrical lead coupled to the needle, and electrical leads coupled to the electrode(s). An example method includes deploying such an injection catheter adjacent to the tissue, extending a needle into the tissue, receiving electrical signals from an electrical lead coupled to the needle and from other electrical leads coupled to the electrode(s), determining a bioelectrical parameter based on the received electrical signals, and determining a depth of the needle inserted into the tissue based on the bioelectrical parameter.

### CROSS-REFERENCE TO RELATED APPLICATIONS

This application claims priority to U.S. Patent Application Ser. No. 63/046,319, filed Jun. 30, 2020, which is hereby incorporated in its entirety by this reference.

### FIELD OF THE DISCLOSURE

The present disclosure generally relates to medical devices and procedures, and more particularly, to methods and apparatus for measuring insertion depth of an injection catheter needle and/or effectiveness of injection into tissue(s).

### BACKGROUND

In the event of end-stage organ failure, approaches such as transplantation of organs (e.g., the heart) may be indicated. However, alternative approaches to repair tissue have been introduced. For example, stem cells or other biologically active solutions may be injected into the damaged tissue. In the case of heart tissue, endocardial or epicardial injection catheters exist to deliver biologically active substances such as stem cells for the purpose of myocardial repair, as opposed to a heart transplant. In this case, the safety and efficacy of such injection depends on the appropriate depth of the injection and delivery of an adequate amount of stem cells into the myocardium.

### SUMMARY

Systems, methods, and devices for measuring injection catheter needle insertion depth and injection efficacy is described herein. In some variations, an injection catheter may comprise a catheter tube, a needle disposed in the catheter tube, an electrode disposed at a distal portion of the catheter tube, a first electrical lead coupled to the needle, and a second electrical lead coupled to the electrode. The needle may be retractable. The needle may be configured to extend from a distal end of the catheter tube into a myocardial tissue of a subject and may be electrically conductive. The first electrical lead and the second electrical lead may be configured to measure a change in bioelectrical parameter. A depth of the needle inserted into the myocardial tissue may be based on the change in the bioelectrical parameter.

In some variations, the first electrical lead traverses a first length of the catheter tube to first proximal portion of the catheter tube. The second electrical lead traverses a second length of the catheter tube to a second proximal portion of the catheter tube. In some variations, the first proximal portion may be the same as the second proximal portion. In some variations, at least one of the first proximal portion or the second proximal portion may be a proximal end of the catheter tube.

In some variations, the injection catheter may further comprise an electrically conductive post coupled between the first electrical lead and the needle. In some variations, the injection catheter may further comprise an electrically conductive post coupled between the first electrical lead and the needle. In some variations, the needle may be hollow.

In some variations, the needle may have at least one hole located at a distal portion of the needle. The at least one hole may provide fluid communication between an interior of the needle and an environment external to the needle. In some variations, the electrode may be disposed on an outer surface of the catheter tube. In some variations, the electrode may be a ring electrode. The change in the bioelectrical parameter may be measured with the needle being in a bipolar configuration.

In some variations, another electrode may be disposed on a part of the subject's body. The other electrode may be a ground electrode and the change in the bioelectrical parameter may be measured with the needle being in a unipolar configuration. In some variations, the injection catheter may further comprise a spring clamp coupled between an outer surface of the needle and the first electrical lead.

In some variations, the injection catheter may further comprise injection tubing disposed in the catheter tube and coupled to a proximal portion of the needle such that an inner volume of the injection tubing may be fluidly coupled to an inner volume of the needle.

In some variations, the injection catheter may further comprise a pressure sensor configured to monitor intraluminal pressure in the catheter tube. In some variations, the catheter tube may comprise a transverse wall that is fixed relative to the distal end of the catheter tube and through which the needle may pass. The injection catheter may further comprise a piston coupled to the needle and a spring coupled between the piston and the transverse wall of the catheter tube. The spring may be configured to compress and the needle may be configured to extend from the distal end of the catheter tube, as the piston may move axially towards the transverse wall of the catheter tube.

In some variations, the bioelectrical parameter may comprise impedance. In some variations, inserting the needle into the myocardial tissue may increase the impedance. A fluid may be injected via the injection catheter into the myocardial tissue when the impedance begins to decrease.

In some variations, a medical system for injecting a fluid into a myocardial tissue may comprise an injection catheter comprising a catheter tube, a needle, an electrode disposed at a distal portion to the catheter tube, a first electrical lead coupled to the needle, and a second electrical lead coupled to the electrode. The needle may be retractable. The needle may be configured to extend from a distal end of the catheter tube into the myocardial tissue of a subject and may be electrically conductive. The medical system may further comprise at least one processor coupled to the first and the second electrical leads configured to: determine a bioelectrical parameter based on electrical signals received from the first and second electrical leads, and determine at least one of: a depth of the needle of the injection catheter inserted into the myocardial tissue based on the bioelectrical parameter, or an amount of the fluid injected from the needle of the injection catheter based on the bioelectrical parameter.

In some variations, the bioelectrical parameter may comprise an impedance, a resistance, a reactance, an inductance, or a capacitance. In some variations, the bioelectrical parameter may comprise an admittance, a conductance, or a susceptance. In some variations, the bioelectrical parameter may comprise a permittivity, a resistivity, or a conductivity.

In some variations, the medical system may further comprise a display. The display may be configured to display at least one of the bioelectrical parameter, the depth of the needle of the injection catheter, or the amount of the fluid injected from the needle.

In some variations, the medical system may further comprise a motor coupled between the processor and the injection catheter. The injection catheter may further comprise a pressure sensor configured to determine an injection pressure. The processor may be further configured to control the motor to automatically extend and retract the needle based on the depth of the needle and the injection pressure.

In some variations, the medical system may further comprise a signal processing circuit coupled between the processor and the first and the second leads. The signal processing circuit may comprise an instrumentation amplifier.

In some variations, a method for injecting fluid into a myocardial tissue of a subject may comprise deploying an injection catheter adjacent to the myocardial tissue. The injection catheter may comprise a catheter tube, a needle disposed in the catheter tube, one or more electrode disposed at a distal portion of the catheter tube, a first electrical lead coupled to the needle, and a second electrical lead coupled to the electrode. The needle may be electrically conductive. The method may also comprise extending the needle from a distal end of the catheter tube into the myocardial tissue, receiving a first electrical signal from the first electrical lead coupled to the needle, receiving a second electrical signal from the second electrical lead coupled to the electrode, determining a bioelectrical parameter based on the received first and second electrical signals, and determining a depth of the needle inserted into the myocardial tissue based on the bioelectrical parameter.

In some variations, the method may further comprise injecting the fluid from the needle of the injection catheter into the myocardial tissue, receiving a third electrical signal from the first electrical lead coupled to the needle, receiving a fourth electrical signal from the second electrical lead coupled to the electrode, determining another bioelectrical parameter based on the received third and fourth electrical signals, and determining an amount of the fluid injected based on the other bioelectrical parameter.

In some variations, the bioelectrical parameter may comprise impedance and injecting the fluid may comprise injecting the fluid when the impedance decreases. In some variations, the bioelectrical parameter may comprise impedance and the needle may be inserted into the myocardial tissue when the impedance increases. In some variations, the needle may be inserted into the myocardial tissue until the impedance decreases.

In some variations, the fluid may comprise stem cells. In some variations, the fluid may comprise a cellular solution. In some variations, the fluid may comprise an acellular solution. In some variations, the bioelectrical parameter may comprise an impedance, a resistance, an inductance, or a capacitance. The bioelectrical parameter may comprise an admittance, a conductance, or a susceptance. In some variations, the bioelectrical parameter may comprise a permittivity, a resistivity, or a conductivity.

In some variations, the tissue may comprise myocardial tissue. In some variations, the method may further comprise sourcing a current to the needle via the first electrical lead or to the electrode via the second electrical lead. The sourced current may comprise a radio frequency (RF) current.

In some variations, the method may further comprise displaying at least one of the bioelectrical parameter or the depth of the needle of the injection catheter. The method may further comprise controlling a motor to automatically extend or retract the needle from the distal end of the catheter tube based on at least the determined depth of the needle.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects.

FIG. 1 is a block diagram of an example medical system having an injection catheter, in accordance with certain aspects of the present disclosure.

FIG. 2 is a cross-sectional view along a longitudinal axis of an example injection catheter capable of measuring one or more bioelectrical parameters, in accordance with certain aspects of the present disclosure.

FIGS. 3A-3D conceptually illustrate different impedances measured by an injection catheter during insertion of a needle into a tissue at different needle insertion depths and release of an injection solution into the tissue, in accordance with certain aspects of the present disclosure.

FIG. 4 is a flow diagram of example operations for injecting fluid into tissue, in accordance with certain aspects of the present disclosure.

### DETAILED DESCRIPTION

Certain aspects of the present disclosure provide methods and apparatus for determining insertion depth of a needle of an injection catheter into target tissue and/or determining an amount of fluid (e.g., a solution containing cellular components such as stem cells, or acellular solutions containing cytokines, genes, viruses, and other proteins) injected by the injection catheter, to provide improved accuracy and safety when injecting the fluid into the tissue with the injection catheter. Such determinations may be based on bioelectrical parameters measured between the electrically conductive needle and one or more electrodes associated with the injection catheter. In this manner, the bioelectrical parameters may be used to make quantitative (or at least semi-quantitative) measurements of needle insertion depth and/or efficacy of the solution injected into the target tissue.

Various aspects of the disclosure are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein, one skilled in the art should appreciate that the scope of the disclosure is intended to cover any aspect of the disclosure disclosed herein, whether implemented independently of or combined with any other aspect of the disclosure. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim.

As used herein, the term “determining” encompasses a wide variety of actions. For example, “determining” may include calculating, computing, processing, deriving, investigating, looking up (e.g., looking up in a table, a database, or another data structure), ascertaining, and the like. Also, “determining” may include receiving (e.g., receiving a signal, data, or information), accessing (e.g., accessing data in a memory), and the like. Also, “determining” may include resolving, selecting, choosing, establishing, and the like.

As used herein, the term “bioelectrical parameter” generally refers to an electrical characteristic of a tissue of a living subject and may include, for example, impedance, resistance, reactance, inductance, capacitance, admittance, conductance, susceptance, permittivity (i.e., dielectric constant), resistivity, conductivity, etc. of the tissue (e.g., myocardial tissue) or liquid (e.g., blood, body fluids, or injected solutions). As used herein, the term “subject” generally refers to a human or another animal, such as a pig or a dog.

### Example Medical System

When injecting a solution into tissue (e.g., myocardial tissue), multiple injections may be performed within the tissue where a proportion of the total injection solution is injected with each injection. It may be prudent for each injection to properly inject the proportion of the solution into the tissue in a repeatable, reliable fashion. During the injection procedure itself, the injection needle of the catheter may be inserted either perpendicular to or obliquely into the endocardial wall. However, the myocardial wall thickness may vary, and may be as thin as 5 mm or less. Full penetration of the myocardium may cause cardiac perforation and may also result in cardiac tamponade or even death. Conventional technologies predetermine the thickness of the myocardial wall so as to calculate a predetermined depth of needle insertion into the myocardium. For instance, myocardial wall thickness may be determined prior to or during a procedure by cardiac imaging modalities such as computed tomography (CT), magnetic resonance imaging (MRI, or echocardiography. The depth of the needle to be inserted into the myocardium can be predetermined based on the output from the cardiac imaging modalities (i.e., based on identifying the thickness of the myocardial wall from the output of the cardiac imaging modalities). An operator (e.g., surgeon, physician, veterinarian, etc.) may prepare the needle to extend to the predetermined depth. The operator may then inject fluid (e.g., solutions containing stem cell or other cellular or acellular components) using the prepared needle. However, there are several challenges associated with this procedure. For example, the operator may not be aware of the fact that an injection needle may have fully penetrated the targeted tissue layers (e.g., the myocardial wall) and entered a cavity (e.g., pericardial space) or that the injection needle may have penetrated other unintended tissue that may render the injection not only ineffective, but also unsafe. Similarly, if the injection occurs before the needle is inserted to an adequate depth into the target tissue, the injected solution may not be injected into the targeted tissue, but instead into a cavity (e.g., ventricular cavity of the heart) or into unintended surrounding tissues, which may render the injection ineffective and may possibly lead to complications from interaction between the injected solution and the unintended tissue.

Accordingly, an injection catheter that can provide real-time feedback information to a system, which an operator can then interpret, is desired. In certain aspects, an injection needle itself may be an electrode. In some examples, the measurements of bioelectrical signals may be performed using the needle as an electrode in a unipolar configuration (e.g., by pairing the needle with a ground electrode on the subject's body), or in a bipolar configuration paired with another electrode (e.g., another electrode associated with the injection catheter). In either the unipolar or bipolar configuration, the electrically conductive needle can be used to measure bioelectrical parameters (e.g., impedance, resistance, reactance, inductance, capacitance, admittance, conductance, susceptance, permittivity (i.e., dielectric constant), resistivity, conductivity, etc.) associated with a tissue (e.g., myocardial tissue). As used herein, being “associated with a tissue” may include not only the tissue itself, but other contributing components considered to be connected in parallel and/or in series between the electrodes (one of which is the electrically conductive needle) and the targeted tissue. As described further herein, the bioelectrical parameter(s) may be measured between a needle at least partially disposed within a target tissue and one or more electrodes located outside of the tissue. When the needle is injected into tissue, the bioelectrical parameters measured with the electrodes may change as the needle is inserted at various depths into the tissue. Accordingly, bioelectrical signals generated and/or measured with an injection catheter having a needle electrode may provide a quantifiable measurement of the depth of insertion, thus improving accurate real-time feedback of the depth of injection needle into the tissue.

In certain aspects, such measurement may also reflect an amount of solution(s) being injected into myocardial tissue. For example, the solution may be injected into the myocardial tissue after determining that the injection needle is at a desired insertion depth. Thereafter, the injection needle may be kept at the desired insertion depth, at least until the needle is retracted. Assessment of solution injection is possible since injected solutions may have different bioelectrical properties than the targeted tissue in which the solution is injected. Once the desired depth of the needle insertion is achieved (and kept relatively unchanged), the bioelectrical parameters measured from the needle electrode, alone or in combination with other electrodes, may reflect the amount of the solution injected into the targeted tissue. For example, cells or non-crystalloids (e.g., proteins, peptides, and other non-cellular biologically active substances or factors) may be injected into myocardial tissues.

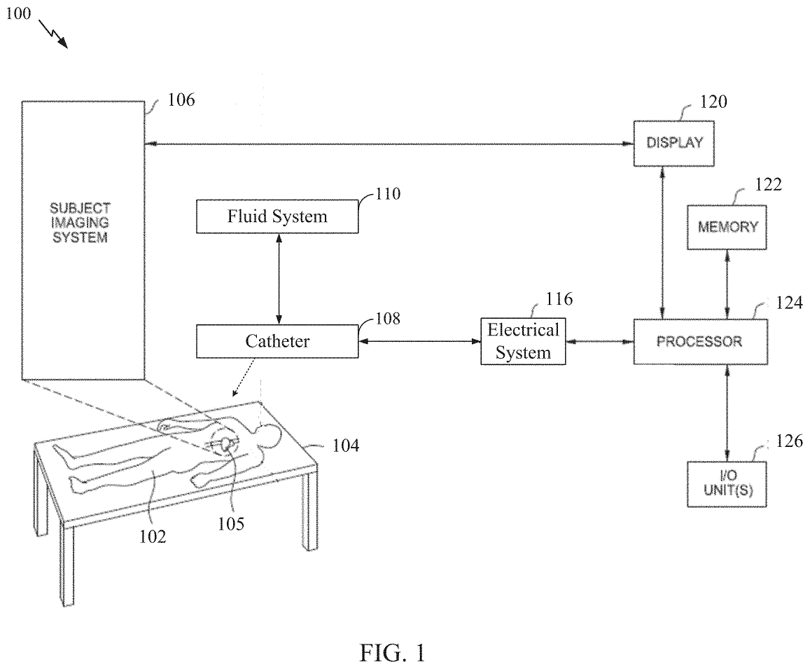

FIG. 1 is a block diagram of an example medical system 100 for determining an insertion depth of a needle in an injection catheter, injecting a fluid (e.g., a solution) from the needle into a tissue, and/or determining an effectiveness of the fluid injection, in accordance with certain aspects of the present disclosure. For example, an injection procedure may deliver any of various suitable treatment solutions into myocardial tissue, as described above. Although myocardial tissue is described herein as an example to facilitate understanding, the reader is to understand that the techniques and apparatus described herein may be provided to other suitable tissues. The medical system 100 may include an injection catheter 108, a fluid system 110, an electrical system 116, a processor 124, memory 122, a display 120, and one or more input/output (I/O) units 126. For certain aspects, the system 100 may also include a table 104 and/or an imaging system 106 (e.g., a 3D electroanatomic mapping system or an ultrasound imaging system for imaging a subject).

For such treatments, a fluid (e.g., a cellular or acellular solution) may be injected into the myocardial tissue. Therefore, the injection catheter 108 may be configured to receive appropriate fluid(s) from the fluid system 110. The fluid system 110 may be a manually operated injection syringe, for example, or in some cases an injection pump and/or reservoir with a control valve. The fluid system 110 may be coupled to the injection catheter 108 by tubing, which may be flexible. In certain aspects, the injection catheter 108 may be inserted into the subject 102 transvascularly through a vein or artery (e.g., of a leg) or epicardially through the chest, of the subject 102. For an injection procedure, it may be desirable to monitor the insertion depth of a needle within myocardial tissue of the heart 105 of the subject 102. The injection catheter 108 may also be in communication with the electrical system 116 such that electrical signals can be acquired and processed and feedback can be generated, as described further herein.

The subject imaging system 106 may include any of various imaging systems (e.g., a 3D electroanatomic mapping system or an ultrasound imaging system) suitable for imaging the targeted tissue or organ(s) (e.g., myocardium of the heart) of the subject 102. For certain aspects, the subject imaging system 106 may include its own display(s). For other aspects, the subject imaging system 106 may output video signals for display on the display 120, as illustrated in FIG. 1. In some cases, the subject imaging system 106 may be able to determine the 3D location of the injection catheter 108 and determine the anatomic geometry of the heart of the subject 102, which can be merged or incorporated with other imaging modalities (e.g., CT scan or MRI scan) to identify the most appropriate site(s) for injection.

The electrical system 116 may include any of various suitable circuits for sourcing signals to the injection catheter 108 and/or processing signals received from the injection catheter, such as a signal acquisition unit. For example, the electrical system 116 may include one or more current sources, filters (analog and/or digital), amplifiers (e.g., isolated preamplifiers and/or an instrumentation amplifier), and/or analog-to-digital converters (ADCs). These circuits may be implemented using discrete components and/or integrated circuits (ICs) to generate and/or process signals. In some cases, the one or more current sources of the electrical system 116 may generate one or more currents that serve as the excitation or test current for determining bioelectrical properties of the target tissue (e.g., radio frequency (RF) low amplitude current that neither excites the target tissue nor generates significant heat). For certain aspects, the electrical system 116 may also be configured to convert one or more optical signals from the injection catheter 108 (e.g., an optical signal from an optical pressure sensor included in the catheter to determine an injection pressure) to an electric signal and process the converted electrical signal accordingly. The electrical system 116 may output signals to the processor 124 and/or receive control signals from the processor 124 to set operating parameters of the electrical system, such as sampling frequency, gain, and/or cutoff frequencies.

For certain aspects, the electrical system 116 may also include a motor (not shown) configured to control insertion depth of the needle in the injection catheter 108, which may be based on measurements of the bioelectrical parameter(s). For example, the motor may be controllable by the processor 124 to automatically extend or retract the needle of the injection catheter 108 based on the depth of the needle and/or the injection pressure. For other aspects, the needle of the injection catheter may be manually extended or retracted. For example, the needle of the injection catheter may be extended by pushing a piston inside the injection catheter and be retracted by the recoil of a spring inside the injection catheter.

For certain aspects, the processor 124 of the medical system 100 may be configured to determine at least one bioelectrical parameter based on signals received from the injection catheter 108 and processed by the electrical system 116. For example, the processor 124 may determine an impedance (Z=R+jX), a resistance (R), a reactance (X), an inductance (L, where X=jωL), and/or a capacitance (C, where X=1/jωC). Additionally, or alternatively, the processor 124 may be configured to determine an admittance (Y=G+jB=1/Z), a conductance (G), a susceptance (B), a permittivity (ε) or other property of a dielectric material, a resistivity (ρ), and/or a conductivity (σ). Utilizing the values of one or more of the measured bioelectrical parameters, the processor 124 may determine an insertion depth of the needle of the injection catheter and/or the amount of solution injected from the needle into the target tissue. The display 120 may be configured, for example, to display the bioelectrical parameter(s), the depth of the needle of the injection catheter 108, and/or the amount of the solution injected from the needle into the target tissue.

The processor 124 may interface with the subject imaging system 106, the electrical system 116, the fluid system 110, the display 120, the memory 122, and/or the I/O unit(s) 126. For example, the processor 124 may read instructions and/or data from the memory 122 and may write data to the memory. For certain aspects, the processor 124 may send control signals to the fluid system 110 (e.g., to a pump in the fluid system to pump fluid through the connecting tubing to the injection catheter and then through an internal lumen of the needle of the injection catheter to one or more exit ports at a distal portion (e.g., the tip) of the needle). For certain aspects, the processor 124 may be able to control manipulation of the injection catheter 108 and/or the needle (e.g., advancing or retracting the needle out of or into of the distal end of the injection catheter via a motor in the injection catheter, as described above). The processor 124 may receive commands and/or input data from the I/O unit(s) 126. The processor may also send control signals and/or output data to the I/O unit(s) 126. For certain aspects, the I/O unit(s) 126 may be utilized to control the motor that drives a motorized fluid system (e.g., a motorized solution injector) and/or another motor that extends and retracts the injection needle of the injection catheter 108.

### Example Injection Catheter

As described above, without feedback signals to provide an operator (e.g., a physician, veterinarian, etc.) with information regarding the insertion depth of a needle, inaccurate injections may occur. For example, a needle insertion that is too shallow may result in ineffective solution injection, and an insertion that is too deep may result in complications, such as perforation, or even death. Thus, an injection catheter as described herein may allow for an operator to assess, in real-time, the depth at which a needle is inserted into targeted tissue (e.g., the myocardium) from the injection catheter. An example injection catheter may include one or more electrodes and one or more electrical leads, as well as a needle that is electrically conductive and functions as an electrode itself. Accordingly, bioelectrical measurements may be taken—via the needle electrode and one or more electrodes associated with the injection catheter itself or with one or more ground electrodes on the subject's body—during a medical procedure to provide an operator with an assessment of how deep the needle is inserted and/or to indicate successful injection of a solution into the target tissue.

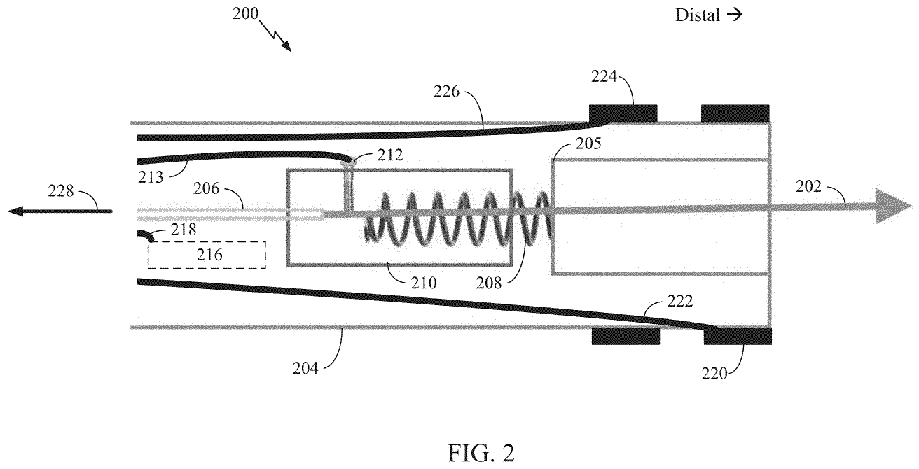

FIG. 2 is a longitudinal cross-sectional view of an example injection catheter 200 for use in injecting fluid (e.g., a solution) into tissue during a medical procedure, in accordance with certain aspects of the present disclosure. The injection catheter 200 is capable of measuring one or more bioelectrical parameters via the injection needle during needle insertion and/or fluid injection into target tissue.

The injection catheter 200 may include a needle 202, catheter shaft 204 (also referred to as “catheter tube”), injection tubing 206, a spring 208, electrodes 220, 224, and electrical leads 213, 222, 226 (e.g., wires). For certain aspects, the injection catheter 200 includes an electrically conductive post 212. The injection catheter 200 may extend leftwards along the arrow 228 to an electrical system (e.g., the electrical system 116) and a processor (e.g., the processor 124) where a bioelectrical parameter may be determined.

The needle 202 may be disposed inside of and extend from the injection tubing 206 within the catheter shaft 204. In certain aspects, the injection tubing 206 may be nonconductive, while the needle 202 may be electrically conductive. For example, the needle may be composed of Nitinol, a metal alloy of nickel and titanium. Additionally, the injection tubing 206 and the needle 202 may be fluidly coupled such that an inner volume of the injection tubing 206 is in fluid communication with an inner volume of the needle 202. In certain aspects, the needle 202 is extendable and retractable for use during operation. As shown, the needle 202 may extend from a distal portion of the catheter shaft 204. In certain aspects, the needle 202 may be hollow. For example, the needle may include at least one hole located at a distal portion (e.g., the tip) of the needle. In this case, the at least one hole may provide fluid communication between the interior of the needle 202 and an environment external to the needle. For example, the environment external to the needle 202 may be tissue, fluid, or a cavity.

In certain aspects, extending and retracting the needle 202 may be governed by at least the spring 208 and the piston 210. The catheter shaft 204 may also include a transverse wall 205 fixed relative to the distal end of the catheter shaft. The needle 202 may be extended by a force exerted (e.g., either manually or by a motor) on a piston 210 configured to travel axially through the lumen in the catheter shaft 204 to compress the spring 208. The needle 202 may be retracted by the recoiling force from the spring 208 once the force is withdrawn. In certain aspects, one end of the spring 208 may be fixed to the catheter shaft 204, and the other end of the spring may be coupled to the needle 202 and/or the piston 210, where the needle and the piston move relative to the catheter shaft 204. For certain aspects, the electrical lead 213 may be coupled directly to the needle 202. For other aspects, a conductive post 212 may be coupled between the needle 202 and the electrical lead 213. For yet other aspects, a clamp (e.g., a metal spring clamp) may be mechanically and electrically coupled between the needle 202 and the electrical lead 213. During operation, the piston 210 may be pushed towards the distal end of the injection catheter 200 to compress the spring 208 and extend the needle 202 from the distal end of the injection catheter 200. Movement of the piston 210 may be continuous or incremental and may be controlled manually or automatically (e.g., by a motor).

In certain aspects, as illustrated, the electrodes 220, 224 may be ring electrodes and may be disposed on an outer surface of the catheter shaft 204. For certain aspects, only one of the electrodes 220, 224 may be included. The electrodes 220, 224 may be coupled to electrical leads 222, 226, respectively. As illustrated, the electrode 220 may be disposed at a distal end of the injection catheter 200, while the electrode 224 may be disposed more proximally. FIG. 2 illustrates two electrodes 220 and 224 solely for illustrative purposes. It should be readily understood that any suitable number of electrodes may be disposed on the injection catheter 200. For example, the injection catheter 200 may include three electrodes. Alternatively, the injection catheter 200 may include four electrodes. In yet other alternative variations, the injection catheter 200 may include no additional electrodes (other than the needle 202).

For certain aspects, the injection catheter 200 may also include one or more pressure sensors 216 located along the injection tubing and/or the needle. The optional pressure sensor(s) 216 may be configured to detect pressure changes (e.g., intraluminal pressure) within the injection catheter 200 during operation (e.g., during a medical procedure) and to provide signals via the lead 218. Sensing the intraluminal pressure using the pressure sensor(s) 216 may serve as a measure of the force applied and/or the mechanical resistance encountered during injection of solution. For example, the pressure sensor 216 may be an electric pressure sensor (e.g., a piezoelectric sensor) or an optical pressure/strain sensor, and the lead 218 may be an electrical lead or an optical fiber, respectively.

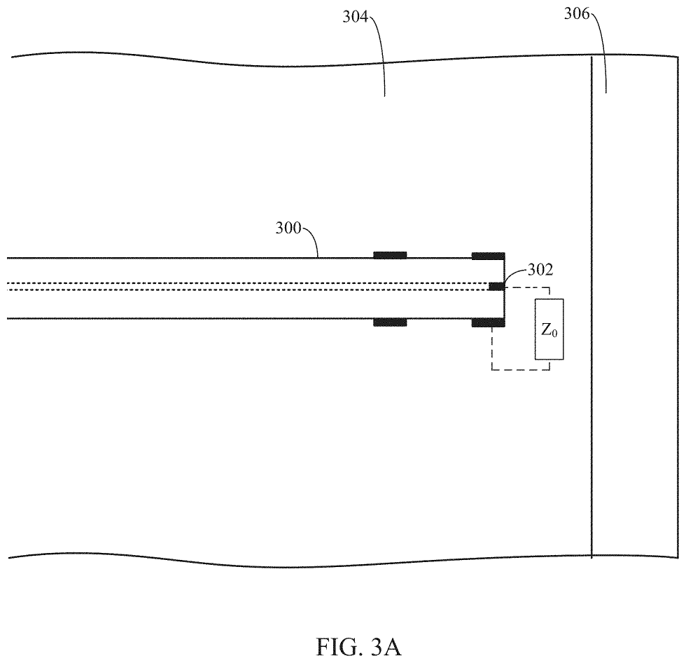

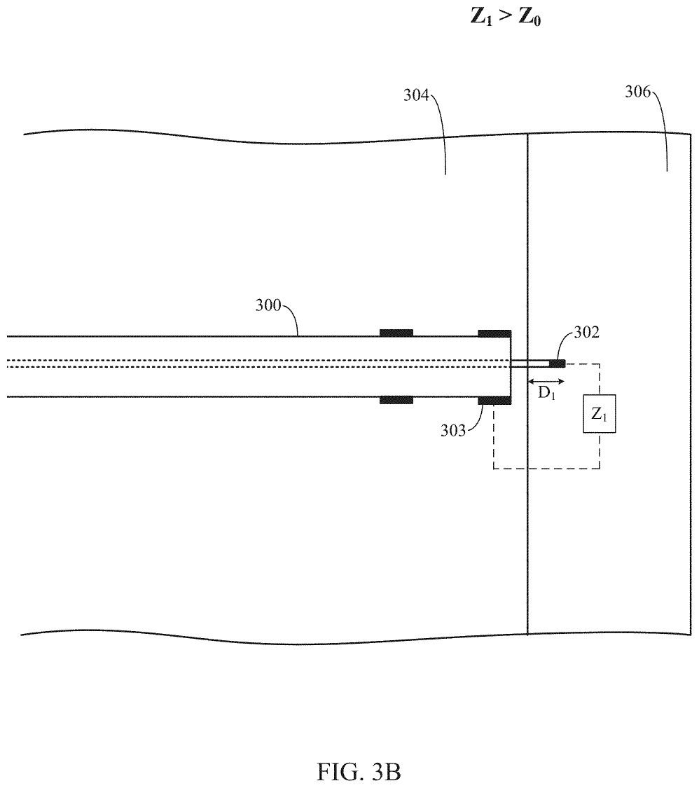

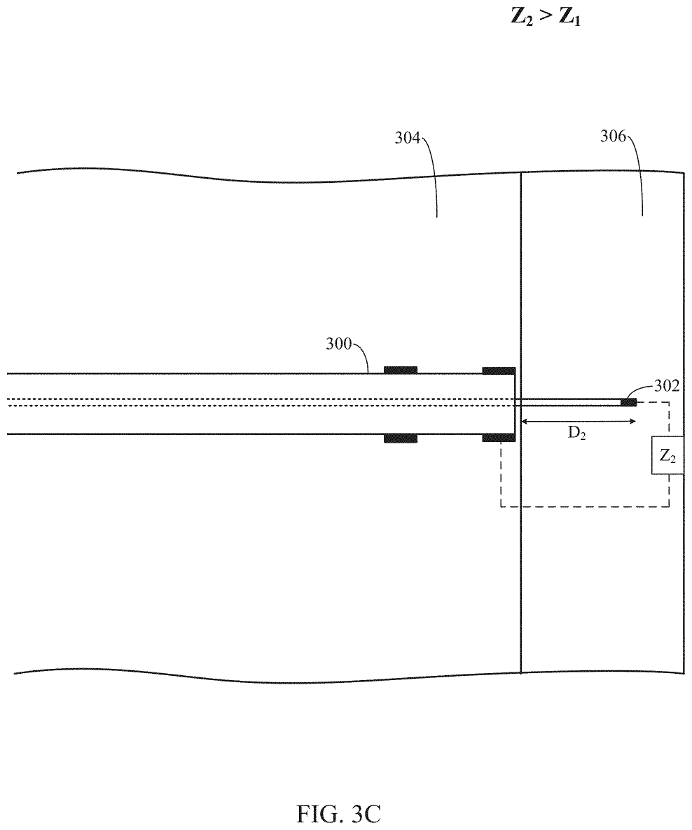

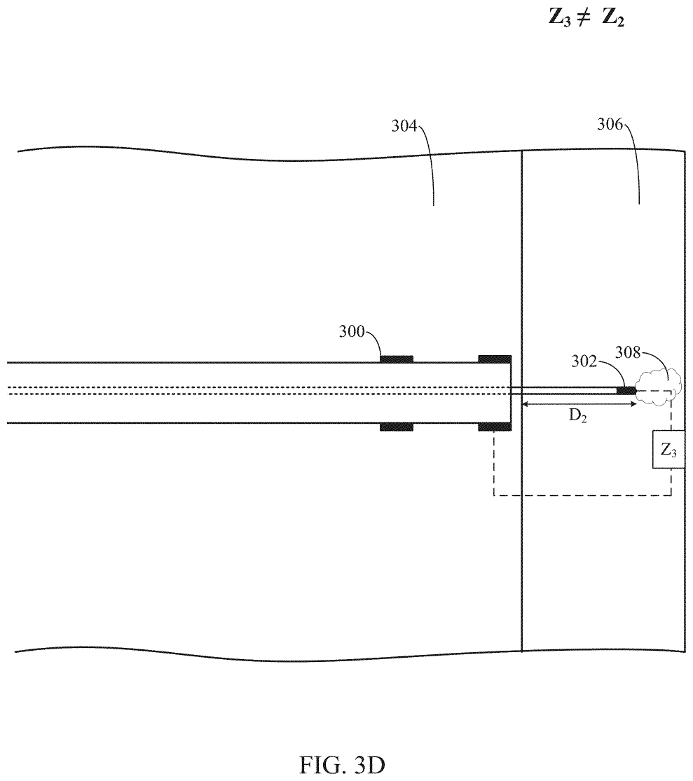

FIGS. 3A-3D conceptually illustrate different impedances measured by an injection catheter 300 (e.g., the injection catheter 200 of FIG. 2) inserted into a vein 304 during insertion of a needle 302 into target tissue 306 with different needle depths and release of a solution 308 or other liquid into the tissue (e.g., during a medical procedure), in accordance with certain aspects of the present disclosure. It should be noted that the sizes depicted may not reflect the relative actual sizes of the injection catheter 300 and the vein 304, but the sizes depicted are for illustrative purposes. Furthermore, the injection catheter 300 depicted may not be shown to include all the components of the injection catheter 200, but the injection catheter 300 may still include such components and have the same design as the injection catheter 200. Although impedance measurements are shown to facilitate understanding of the concepts, the reader is to understand that alternative or additional bioelectrical parameters may be measured.

As shown in FIG. 3A, the injection catheter 300 may be disposed in a vein 304 and adjacent to tissue 306. A needle 302 (e.g., the needle 202 of FIG. 2) may be retracted within the injection catheter 300 in the operational stage depicted. At this stage, the impedance (Z₀) of the blood in the vein may be measured by the system, which may be relatively low and may be similar to the impedance of saline solution, for example. In some variations, the impedance measurement may be performed using the needle 302 in a unipolar configuration. For example, the needle 302 may be electrically coupled to a ground electrode placed on the body of a subject. For example, the ground electrode may be placed on a patch affixed to a part of the body of the subject. In some variations, the impedance measurement may be performed using the needle 302 in a bipolar configuration by pairing the needle 302 with another electrode (e.g., electrode 220 and/or electrode 224 in FIG. 2) on the injection catheter 300.

As depicted in FIG. 3B, the needle 302 may be extended from a distal end of the injection catheter 300 and inserted into the tissue 306 to a first depth D₁. The needle 302, being electrically conductive, may function as an electrode with which an electrical system (e.g., the electrical system 116 of FIG. 1) may measure a bioelectrical parameter between the needle 302 and an electrode 303. For example, inserting the needle to the depth D₁ may lead to the system measuring an impedance Z₁, where Z₁>Z₀. An operator, during operation, may be able to determine based on readings provided by a display (e.g., the display 120) to insert the needle 302 further into the tissue 306. For example, the measured impedance Z₁ (and corresponding depth D₁) may not be as large (or deep) as desired.

As shown in FIG. 3C, the needle 302 may be inserted further into the tissue 306 to a depth D₂ (>D₁), and the corresponding impedance Z₂ may be measured. In this case, due to the greater tissue depth, the impedance Z₂ may be greater than the impedance Z₁. If the operator extends the needle 302 too far into the tissue 306, the impedance may register as being lower than Z₁, indicating that the needle penetrated through the tissue 306, whereas it is generally desired for the needle to remain in the tissue 306 for delivering the fluid to the desired site within the tissue.

As shown in FIG. 3D, the operator may determine the needle 302 is inserted to an appropriate depth, and proceed to inject a solution 308 (e.g., a stem cell solution) into the tissue 306. Injecting the solution 308 into the tissue 306 may produce a different impedance Z₃, which may be different from the impedance Z₂. For example, injecting solution 308 with cellular content (e.g., a stem cell solution) may produce an impedance Z₃ which may be greater than the impedance Z₂. Alternatively, injecting solution 308 with non-cellular content may produce an impedance Z₃ which may be lesser than the impedance Z₂. In other words, the change in impedance (or other bioelectrical parameter) may indicate the amount of solution 308 injected into the tissue 306.

### Example Operations for Fluid Injection

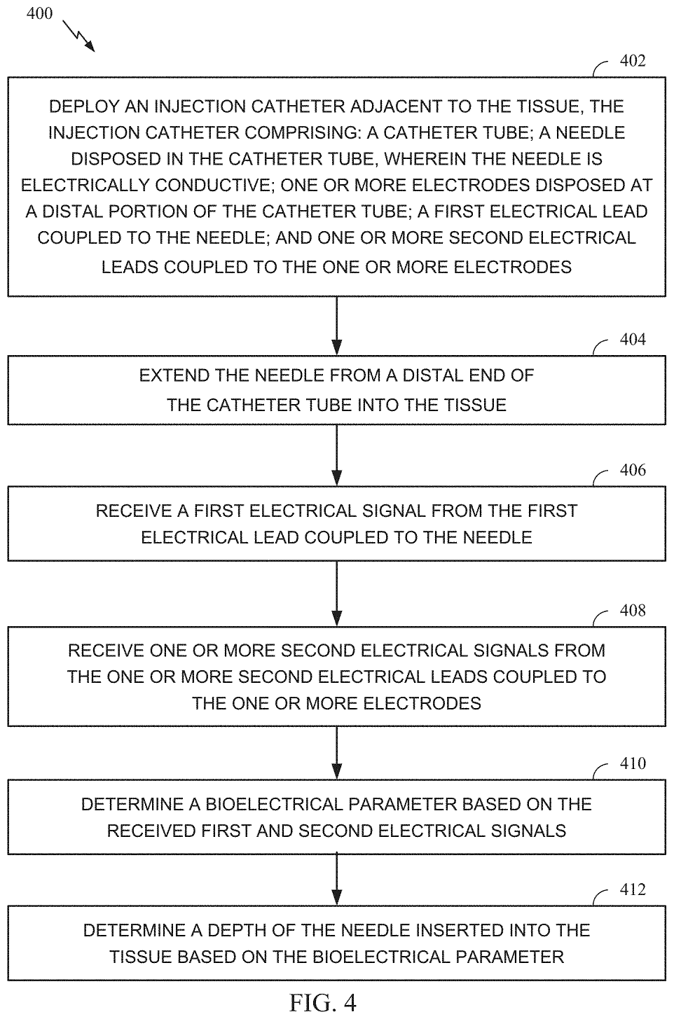

FIG. 4 is a flow diagram of example operations 400 for injecting a fluid (e.g., a solution) into a target tissue (e.g., myocardial tissue), in accordance with certain aspects of the present disclosure. At least some of the operations 400 may be performed by a medical system, such as the medical system 100 of FIG. 1. In some cases, some of the operations 400 may be performed by at least one operator (e.g., a physician, a veterinarian, and/or other medical staff). In certain aspects, the fluid is a cellular solution (which may include stem cells, for example) or an acellular solution.

The operations 400 may begin, at block 402, after the subject (e.g., subject 102) is sedated if desired for the procedure. At block 402, the operator and/or the system may deploy an injection catheter (e.g., the injection catheter 200) adjacent to the target tissue. The injection catheter may include a catheter tube (e.g., the catheter shaft 204) and a needle (e.g., the needle 202) disposed in the catheter tube. The needle may be retractable, may be configured to extend from a distal end of the catheter tube, and may be electrically conductive. The injection catheter may also include one or more electrodes (e.g., the electrodes 220, 224) disposed at a distal portion of the catheter tube, a first electrical lead (e.g., the electrical lead 213) coupled to the needle, and one or more second electrical leads (e.g., the electrical leads 222, 226) coupled to the one or more electrodes. For certain aspects, deployment and movement (e.g., deflection) of the catheter may be performed by manipulating a first handle of the injection catheter.

At block 404, the operator and/or the system may extend the needle from a distal end of the catheter into the tissue. For certain aspects, extension and/or retraction of the needle from the distal end of the catheter may be performed manually (e.g., by manipulating a second handle of the injection catheter. For other aspects, extension and/or retraction of the needle may be controlled automatically (e.g., by a motor).

At block 406, the system may receive a first electrical signal from the first electrical lead coupled to the needle. At block 408, the system may receive one or more second electrical signals from the one or more second electrical leads coupled to the one or more electrodes (e.g., electrodes on the injection catheter and/or electrodes positioned on a portion of the subject's body), which may be configured for unipolar or bipolar recordings. For example, each of the first and second electrical signals may be a voltage signal, such as a low amplitude radio frequency (RF) voltage signal.

At block 410, the system may determine a bioelectrical parameter based on the received first and second electrical signals. For example, the bioelectrical parameter may include an impedance, a resistance, a reactance, an inductance, a capacitance, an admittance, a conductance, a susceptance, a permittivity, a resistivity, a conductivity, etc.

At block 412, the system may determine a depth of the needle inserted into the tissue based on the bioelectrical parameter.

In certain aspects, the operator and/or the system may inject the fluid from the needle of the injection catheter into the tissue. In this case, the system may receive a third electrical signal from the first electrical lead coupled to the needle, receive one or more fourth electrical signals from the one or more second electrical leads coupled to the one or more electrodes, determine another bioelectrical parameter based on the received third and fourth electrical signals, and determine an amount of the fluid (effectively) injected into the target tissue based on the other bioelectrical parameter. For certain aspects, the amount of (effectively) injected fluid may be determined based on changes in the other bioelectrical parameter or a difference between the other bioelectrical parameter and the bioelectrical parameter. The determination of the fluid amount may be quantitative or semi-quantitative.

In certain aspects, the system may source a current to the needle via the first electrical lead or to at least one of the electrodes via at least one of the second electrical leads. For other aspects, the system may source a current to the needle or to at least one of the electrodes via an electrical lead other than the first and second electrical leads. In either case, the sourced current may be, for example, an AC current, such as a high frequency (e.g., RF) current at a relatively low amplitude that neither electrically excites the target tissue nor generates significant heat, but serves as the exciting or test current for determining bioelectrical properties of the target tissue.

In certain aspects, the system may display at least one of the bioelectrical parameter or the insertion depth of the needle of the injection catheter.

According to certain aspects, the system may control a motor to automatically extend or retract the needle from the distal end of the catheter tube based on at least the determined insertion depth of the needle.

Certain aspects of the present disclosure provide a system (e.g., the medical system 100) for injecting fluid (e.g., a solution) into target tissue (e.g., tissue of a subject during a medical procedure). The system includes an injection catheter as described herein. The system also includes at least one processor (e.g., the processor 124) coupled to the first and second electrical leads and generally configured to determine a bioelectrical parameter based on electrical signals and to determine at least one of: (a) a depth of the needle of the injection catheter inserted into the tissue based on the bioelectrical parameter or (b) an amount of the fluid injected from the needle of the injection catheter into the tissue based on the bioelectrical parameter.

According to certain aspects, the bioelectrical parameter includes an impedance, a resistance, a reactance, an inductance, a capacitance, an admittance, a conductance, a susceptance, a permittivity, a resistivity, or a conductivity.

In certain aspects, the system may include a display, wherein the display is configured to display at least one of the bioelectrical parameter, the depth of the needle of the injection catheter into the tissue, or the amount of the fluid injected from the needle into the tissue.

According to certain aspects, the system may include a motor coupled between the processor and the injection catheter. In this case, the injection catheter may further include a pressure sensor (e.g., the pressure sensor 216) configured to determine an injection pressure, and the processor is further configured to control the motor to automatically extend or retract the needle based on the depth of the needle and the injection pressure.

In certain aspects, the system may include a signal processing circuit (e.g., the electrical system 116) coupled between the processor and the first and second electrical leads. The signal processing circuit may include an instrumentation amplifier, for example.

### CONCLUSION

Certain aspects of the present disclosure use the needle of an injection catheter as an electrode—either in a unipolar manner or a bipolar fashion with another electrode associated with the catheter—to measure one or more bioelectrical parameters, such as impedance, resistance, reactance, inductance, capacitance, admittance, conductance, susceptance, permittivity, resistivity, conductivity, and the like. These bioelectrical parameters may change as the needle is inserted into the target tissue (e.g., myocardial tissue) and/or as an injection solution is injected from the needle into the tissue. With calibration, changes in the bioelectrical parameters may provide a quantitative or semi-quantitative measurement of the depth of insertion or the amount of solution injected.

Any of the operations described above, such as the operations 400, may be included as instructions in a computer-readable medium for execution by a processing system. The (non-transitory) computer-readable medium may comprise any suitable memory or other storage device for storing instructions, such as read-only memory (ROM), random access memory (RAM), flash memory (e.g., USB flash drive), an electrically erasable programmable ROM (EEPROM), a compact disc ROM (CD-ROM), a floppy disk, or a digital versatile disc ROM (DVD-ROM).

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

As used herein (including the claims that follow), a phrase referring to “at least one of” a list of items refers to any combination of those items, including single members. As an example, “at least one of: x, y, and z” is intended to cover: x, y, z, x-y, x-z, y-z, x-y-z, and any combination thereof (e.g., x-y-y and x-x-y-z).

While the foregoing is directed to certain aspects of the present disclosure, other and further aspects may be devised without departing from the basic scope thereof, and the scope thereof is determined by the claims that follow.

What is claimed is:

1. An injection catheter comprising:
a catheter tube;
a needle disposed in the catheter tube, wherein the needle is retractable, is configured to extend from a distal end of the catheter tube into a myocardial tissue of a subject, and is electrically conductive;
an electrode disposed at a distal portion of the catheter tube;
a first electrical lead coupled to the needle; and
a second electrical lead coupled to the electrode,
wherein the first electrical lead and the second electrical lead are configured to measure a change in a bioelectrical parameter, and
wherein a depth of the needle inserted into the myocardial tissue is based on the change in the bioelectrical parameter.

2. The injection catheter of claim 1, wherein the first electrical lead traverses a first length of the catheter tube to a first proximal portion of the catheter tube.

3. The injection catheter of claim 2, wherein the second electrical lead traverses a second length of the catheter tube to a second proximal portion of the catheter tube.

4. The injection catheter of claim 3, wherein the first proximal portion is the same as the second proximal portion.

5. The injection catheter of claim 3, wherein at least one of the first proximal portion or the second proximal portion is a proximal end of the catheter tube.

6. The injection catheter of claim 1, further comprising an electrically conductive post coupled between the first electrical lead and the needle.

7. The injection catheter of claim 1, wherein the needle is hollow.

8. The injection catheter of claim 1, wherein the needle has at least one hole located at a distal portion of the needle, the at least one hole providing fluid communication between an interior of the needle and an environment external to the needle.

9. The injection catheter of claim 1, wherein the electrode is disposed on an outer surface of the catheter tube.

10. The injection catheter of claim 9, wherein the electrode is a ring electrode.

11. The injection catheter of claim 9, wherein the change in the bioelectrical parameter is measured with the needle being in a bipolar configuration.

12. The injection catheter of claim 1, wherein another electrode is disposed on a part of the subject's body.

13. The injection catheter of claim 12, wherein the other electrode is a ground electrode and the change in the bioelectrical parameter is measured with the needle being in a unipolar configuration.

14. The injection catheter of claim 1, further comprising a spring clamp coupled between an outer surface of the needle and the first electrical lead.

15. The injection catheter of claim 1, further comprising injection tubing disposed in the catheter tube and coupled to a proximal portion of the needle, such that an inner volume of the injection tubing is fluidly coupled to an inner volume of the needle.

16. The injection catheter of claim 1, further comprising a pressure sensor configured to monitor intraluminal pressure in the catheter tube.

17. The injection catheter of claim 1, wherein the catheter tube comprises a transverse wall that is fixed relative to the distal end of the catheter tube and through which the needle passes.

18. The injection catheter of claim 17, further comprising:
a piston coupled to the needle; and
a spring coupled between the piston and the transverse wall of the catheter tube.

19. The injection catheter of claim 18, wherein the spring is configured to compress and the needle is configured to extend from the distal end of the catheter tube, as the piston is moved axially towards the transverse wall of the catheter tube.

20. The injection catheter of claim 1, wherein the bioelectrical parameter comprises impedance.

21. The injection catheter of claim 20, wherein inserting the needle into the myocardial tissue increases the impedance.

22. The injection catheter of claim 21, wherein a fluid is injected via the injection catheter into the myocardial tissue when the impedance begins to decrease.

23. A medical system for injecting a fluid into a myocardial tissue, the medical system comprising:
an injection catheter comprising:
a catheter tube;
a needle disposed in the catheter tube, wherein the needle is retractable, is configured to extend from a distal end of the catheter tube into the myocardial tissue of a subject, and is electrically conductive;
an electrode disposed at a distal portion of the catheter tube;
a first electrical lead coupled to the needle; and
a second electrical lead coupled to the electrode; and
at least one processor coupled to the first and second electrical leads and configured to:
determine a bioelectrical parameter based on electrical signals received from the first and second electrical leads; and
determine at least one of:
a depth of the needle of the injection catheter inserted into the myocardial tissue based on the bioelectrical parameter; or
an amount of the fluid injected from the needle of the injection catheter based on the bioelectrical parameter.

24. The medical system of claim 23, wherein the bioelectrical parameter comprises an impedance, a resistance, a reactance, an inductance, or a capacitance.

25. The medical system of claim 23, wherein the bioelectrical parameter comprises an admittance, a conductance, or a susceptance.

26. The medical system of claim 23, wherein the bioelectrical parameter comprises a permittivity, a resistivity, or a conductivity.

27. The medical system of claim 23, further comprising a display, wherein the display is configured to display at least one of the bioelectrical parameter, the depth of the needle of the injection catheter, or the amount of the fluid injected from the needle.

28. The medical system of claim 23, further comprising a motor coupled between the processor and the injection catheter, wherein:
the injection catheter further comprises a pressure sensor configured to determine an injection pressure; and
the processor is further configured to control the motor to automatically extend or retract the needle based on the depth of the needle and the injection pressure.

29. The medical system of claim 23, further comprising a signal processing circuit coupled between the processor and the first and second electrical leads, wherein the signal processing circuit comprises an instrumentation amplifier.

30. A method for injecting a fluid into a myocardial tissue of a subject, the method comprising:
deploying an injection catheter adjacent to the myocardial tissue, the injection catheter comprising:
a catheter tube;
a needle disposed in the catheter tube, wherein the needle is electrically conductive;
one or more electrodes disposed at a distal portion of the catheter tube;
a first electrical lead coupled to the needle; and
a second electrical lead coupled to the electrode;
extending the needle from a distal end of the catheter tube into the myocardial tissue;
receiving a first electrical signal from the first electrical lead coupled to the needle;
receiving a second electrical signal from the second electrical lead coupled to the electrode;
determining a bioelectrical parameter based on the received first and second electrical signals; and
determining a depth of the needle inserted into the myocardial tissue based on the bioelectrical parameter.

31. The method of claim 30, further comprising:
injecting the fluid from the needle of the injection catheter into the myocardial tissue;
receiving a third electrical signal from the first electrical lead coupled to the needle;
receiving a fourth electrical signal from the second electrical lead coupled to the electrode;
determining another bioelectrical parameter based on the received third and fourth electrical signals; and
determining an amount of the fluid injected based on the other bioelectrical parameter.

32. The method of claim 31, wherein the bioelectrical parameter comprises impedance and injecting the fluid comprises injecting the fluid when the impedance decreases.

33. The method of claim 30, wherein the bioelectrical parameter comprises impedance and wherein the needle is inserted into the myocardial tissue when the impedance increases.

34. The method of claim 33, wherein the needle is inserted into the myocardial tissue until the impedance decreases.

35. The method of claim 30, wherein the fluid comprises stem cells.

36. The method of claim 30, wherein the fluid comprises a cellular solution.

37. The method of claim 30, wherein the fluid comprises an acellular solution.

38. The method of claim 30, wherein the bioelectrical parameter comprises an impedance, a resistance, a reactance, an inductance, or a capacitance.

39. The method of claim 30, wherein the bioelectrical parameter comprises an admittance, a conductance, or a susceptance.

40. The method of claim 30, wherein the bioelectrical parameter comprises a permittivity, a resistivity, or a conductivity.

32. The method of claim 30, wherein the tissue comprises myocardial tissue.

41. The method of claim 30, further comprising sourcing a current to the needle via the first electrical lead or to the electrode via the second electrical lead.

42. The method of claim 41, wherein the sourced current comprises a radio frequency (RF) current.

43. The method of claim 30, further comprising displaying at least one of the bioelectrical parameter or the depth of the needle of the injection catheter.

44. The method of claim 30, further comprising controlling a motor to automatically extend or retract the needle from the distal end of the catheter tube based on at least the determined depth of the needle.
